(19) United States
(12) Patent Application Publication
(10) Pub. No.: US 2015/0044296 A1
(43) Pub. Date: Feb. 12, 2015

(54) COMBINATION THERAPY TO IMPROVE JOINT, TENDON, AND LIGAMENT HEALING

(71) Applicants: Leonard B. MILLER, Brookline, MA (US); Stephen WARREN, New York, NY (US)

(72) Inventors: Leonard B. Miller, Brookline, MA (US); Stephen Warren, New York, NY (US)

(21) Appl. No.: 14/386,390

(22) PCT Filed: Mar. 15, 2013
(86) PCT No.: PCT/US2013/032138

§ 371 Date: Sep. 19, 2014

Related U.S. Application Data
(60) Provisional application No. 61/613,734, filed on Mar. 21, 2012.

(51) Int. Cl. A61K 38/29; A61K 9/16; A61K 45/06; A61K 31/395

(52) U.S. Cl. CPC A61K 38/29; A61K 31/395; A61K 9/16; A61K 45/06
USPC 424/501; 514/11.8

(57) ABSTRACT

The present invention is directed to kit, drug combinations and methods for promoting endogenous bone marrow (BM)-derived vasculogenic progenitor cell (PC) mobilization, sensitization of such cells and chemotaxis to sites of joint injury or disease. One embodiment of the present invention, directed to a method of promoting joint complex healing, comprises the step of administering an effective amount of a bone marrow (BM) derived vasculogenic progenitor cell mobilization factor to an animal or human exhibiting joint injury or joint disease. The method further comprises the step of administering, concurrently to the mobilization factor, an effective amount of a progenitor cell sensitizing factor to mobilize progenitor cells and sensitize the progenitor cells to one or more chemotactic agents present at the site of joint injury or joint disease.

### BACKGROUND OF THE INVENTION

Injury to the joint complex in healthy humans can takes months to heal. Furthermore, in certain types of injury (e.g. meniscal or ligament tear, avascular necrosis, etc.), a substantial proportion of bony defects do not heal. Current therapeutic options for the treatment of joint injuries include splints, casts, and arthroscopic surgery to treat floating cartilage, torn surface cartilage, ligament reconstruction, and trim damaged cartilage. While the above treatment options can be somewhat successful, they are associated with complications such as hemarthrosis, infection, thromboembolic disease, anesthetic complications, reflex sympathetic dystrophy, iatrogenic ligament injury, iatrogenic fracture, and neurologic injuries. Moreover, the above treatment options do not improve or augment the body's own endogenous repair mechanisms. To avoid the above complications and improve the joint healing process new therapeutic options are necessary.

### SUMMARY OF THE INVENTION

Accordingly, to overcome these challenges, the present invention encompasses, in part, promotion of endogenous bone marrow (BM)-derived vasculogenic progenitor cell (PC) mobilization, sensitization of such cells and chemotaxis to sites of injury using therapeutics or combinations of therapeutics.

During the joint complex (e.g. bone, cartilage, tendon, and ligament) healing process, an adequate blood supply is critical for successful bone, cartilage, tendon, and ligament regeneration. Our recent studies have demonstrated that signals from the site of tissue injury can mobilize bone marrow (BM)-derived vasculogenic progenitor cells (PCs) into the peripheral circulation and recruit these vasculogenic PCs to the injury site where they contribute to neovascularization, tissue repair and regeneration. While we have shown that vasculogenic PC levels in the peripheral blood of humans and mice naturally increase after injury, we have also demonstrated that augmenting this natural response mechanism can dramatically improve healing. Further we have shown that small molecule-mediated mobilization of vasculogenic PCs results in increased trafficking of these PCs to the injury site, increased new blood vessel formation, and increase speed of tissue healing.

One embodiment of the present invention, directed to a method of promoting joint complex healing, comprises the step of administering an effective amount of a bone marrow (BM)-derived vasculogenic progenitor cell mobilization factor to an animal or human exhibiting joint injury or joint disease. The method further comprises the step of administering, concurrently to the mobilization factor, an effective amount of a progenitor cell sensitizing factor to mobilize progenitor cells and sensitize the progenitor cells to one or more chemotactic agents present at the site of joint injury or joint disease.

As used herein, the term “bone marrow derived vasculogenic progenitor cell” is used as it is used in the medical and biological sciences to denote one or more stem cells which have their site of origin in the bone marrow and are released into the blood stream. This discussion will sometimes use the abbreviation “BM PC” for such term. The term “mobilization factor” is used to denote a compound or group of compounds that cause BM PCs to be released from the bone marrow into the circulation. The term “sensitizing factor” is used to denote one or more compounds which cause BM PCs to be responsive to chemotactic agents which are released by injured tissue and cause migration of BM PC to the site of injury. A chemotactic agent is a compound or group of compounds which promote the migration of BM PCs to a site of injury. As used herein, the term “joint” is used to denote the bone, cartilage, tendon and other tissues in close proximity to a flexible union of two or more bones.

One embodiment of the present method features a mobilization factor selected from the group consisting of CXCR4 agonists and partial agonists, granulocyte stimulating factor (G-CSF),granulocyte-macrophage stimulating factor (GM-CFS), interleukin-1 (11-1), interleukin-3 (Il-3), interleukin-8 (Il-8), PIXY-321 (GM-CSF/Il-3 fusion protein), macrophage inflammatory protein, growth related oncogene and agents and factors that modify the expression of the above factors, for example without limitation, siRNA to a repressor of the above agent.

Examples of CXCR4 agonists and partial agonists are disclosed in U.S. Pat. No. 7,935,692 B2, which is incorporated by reference herein. AMD3100 is one compound which is disclosed in the '692 patent and is sold under the trademark PLERIXAFOR ® (Genzyme, Boston, Mass.).

One embodiment of the present method features a sensitizing factor is selected from the group consisting of parathyroid hormone and subunits of such hormone, NEL-like molecule-1, calreticulin and closely related molecules, and agents and factors that modify the expression of the above factors, such as by way of example without limitation, siRNA to a repressor of the above agent. On example of such a hormone is, without limitation, recombinant human parathyroid hormone, known as teriparatide and sold under the trademark, FORTEO®; (Eli Lilly and Company, Indianapolis, Ind.).

Concurrent administration means at or about the same time. The concurrent administration may be performed in a single occurrence or multiple occurrences over time.

One embodiment of the present method features a further step of administering at least one chemotactic factor to the area of the joint injury or joint disease. Examples of chemotactic agents include, without limitation, transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors, such as by way of example, without limitation, siRNA directed to repressor of the above agent. The detailed discussion that follows features the stromal derived growth factor, stromal cell derived factor-1 (SDF-1),

In one embodiment of the present method the mobilization factor and sensitization factor are co-administered by subcutaneous, intraperitoneal or intravenous injection. However, other modes of administration may be used including by way of example, without limitation, oral, sublingual, buccal, rectal, nasal, transdermal and pulmonary administration.

One embodiment features a chemotactic agent is administered to the site of injury or to the site of joint disease to one or more of the soft tissues proximal to the injury. The administration can be by spray, or washing with solutions loaded with such chemotactic agent or by direct injection. One embodiment of the present invention features incorporation of the chemotactic agent into a biopolymer which over time releases the chemotactic agent. As used herein, the term biopolymer refers to a polymer that is broken up and or consumed by the body in which it is placed by natural processes. Examples of a biopolymer include, without limitation, gelatin, polyglyconic and polylactic acid derivatives.

A further embodiment of the present invention is directed to an article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at the site of joint injury or joint disease.

One example, without limitation, of the dosage form features an effective amount of the mobilization factor and an effective amount of the sensitizing factor lyophilized and held in a vial for reconstitution, or in a vial in solution form.

A further embodiment of the dosage form comprises an effective amount of the mobilization factor and an effective amount of the sensitizing factor held in a package with an effective amount of a chemotactic agent in the form of a kit. The chemotactic agent is administered to a disease joint or an injured joint to direct mobilized and sensitized progenitor cells to the site where healing is desired. Preferably, the kit includes instructions and other materials and tools for making and using the elements contained therein.

For example, without limitation, the dosage form in the form of a kit may comprise a chemotactic agent lyophilized and held in a vial for reconstitution. In the event the chemotactic agent is administered by direct injection to soft tissue in and around the injured joint or disease joint, the kit may comprise an injection needle and syringe. Other embodiments feature a chemotactic agent held in a sustained release vehicle, for example, a sustained release vehicle such as a biopolymer. Examples of biopolymers include gelatin, polyglyconic and polylactic acid derivatives. The biopolymers can be administered as microspheres or implants.

The use of a combination of mobilization factors and sensitizing factors improves healing of joint injuries and joint disease over healing exhibited by the use of either factor separate and apart from the other.

Other features, objects, and advantages of the invention will be apparent to those skilled in the art upon viewing the drawings which are described briefly below and reading the detailed description that follows. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWING

FIG. 1 depicts a dosage form incorporated into a kit embodying features of the present invention.

### DETAILED DESCRIPTION OF THE INVENTION

Embodiment of the present invention will now be described in detail with respect to an article of manufacture, a therapeutic dosage form reflecting the preferred embodiments of the invention. Those skilled in the art will recognize that the details of such dosage form can be modified and altered and what is thought to be preferred embodiments may change over time. Therefore the present discussion should not be considered limiting.

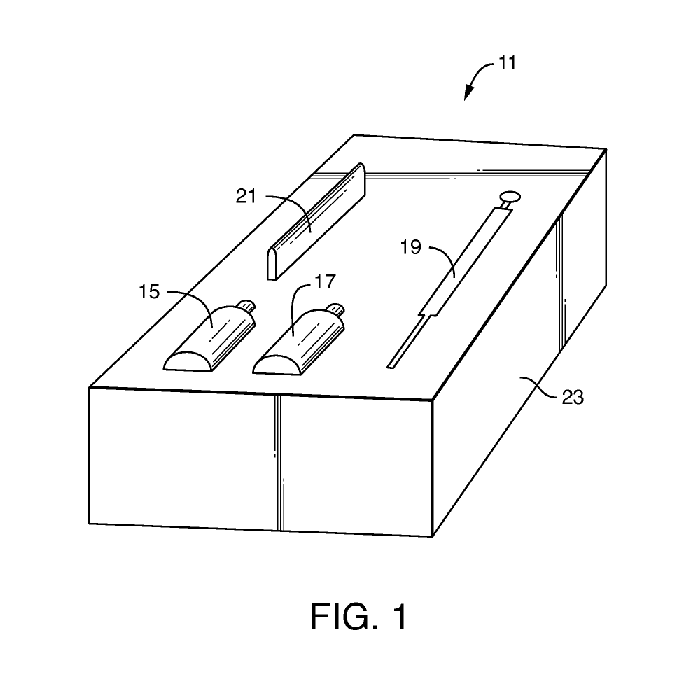

Turning now to FIG. 1, a kit embodying features of the present invention, generally designated by the numeral 11, is depicted. The kit has the following major components, a first vial 15, a second vial 17, a syringe 19, instruction for use 21 and packaging in the form of a box 23. Although a box 23 is depicted, suitable packaging may take many forms. For example, without limitation, suitable packaging may comprise bags, plastic or paper wraps, bundles and the like known in the art. The box 23 is preferably fitted with a cover [not shown] to provide a more complete contained enclosure.

First vial 15 containing at least two compounds, bone marrow (BM)-derived vasculogenic progenitor cell mobilization factor and progenitor cell sensitizing factor. The mobilization factor is selected from the group consisting of CXCR4 agonists and partial agonists, granulocyte stimulating factor (G-CSF),granulocyte-macrophage stimulating factor (GM-CFS), interleukin-1 (Il-1), interleukin-3 (Il-3), interleukin-8 (Il-8), PIXY-321 (GM-CSF/Il-3 fusion protein),macrophage inflammatory protein, and growth related oncogene and agents and factors that modify the expression of the above factors, for example without limitation, siRNA to a repressor of the above agent. For the purpose of this discussion, the mobilization factor is a CXCR4 partial agonist, AMD3100, disclosed in the '692 patent and is sold under the trademark PLERIXAFOR ® (Genzyme, Boston, Mass.).

The sensitizing factor is selected from the group consisting of parathyroid hormone and subunits of such hormone, NEL-like molecule-1, calreticulin and closely related molecules, and agents and factors that modify the expression of the above factors such as by way of example without limitation, siRNA to a repressor of the above agent. For the purpose of this discussion, the sensitizing factor is recombinant human parathyroid hormone, known as teriparatide and sold under the trademark, FORTEO® (Eli Lilly and Company, Indianapolis, Ind.).

The two compounds are held as lyophilized powders for reconstitution in first vial 15. Upon reconstitution, the powders form a solution for injection in which an injection will administer AMD3100 (approximately 8-12 mg/kg of weight of individual or animal) and teriparatide (approximately 0.228-0.342 mcg/kg of weight of individual or animal). These amounts represent an effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at the site of joint injury or joint disease.

These effective amounts of bone marrow (BM)-derived vasculogenic progenitor cell mobilization factor and progenitor cell sensitizing factor are administered to the individual or animal by subcutaneous, intraperitoneal, intramuscular injection by syringe 19. However, other means for providing concurrent administration of the mobilization factor and sensitizing factor may be used including, by way of example, without limitation, oral, sublingual, buccal, nasal, pulmonary, rectal, transdermal and ocular administration.

The second vial 17 containing a chemotactic agent lyophilized for reconstitution. Examples of chemotactic agents include, without limitation, transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors, such as by way of example, without limitation, siRNA directed to repressor of the above agent. For the purpose of this discussion, the chemotactic agent is stromal cell derived factor-1 (SDF-1). SDF-1 is administered in an amount ranging from 1.00 ng to about 100 ng. In the event the chemotactic agent is administered by direct injection to soft tissue in and around the injured joint or disease joint, the kit 11 may comprise a second injection needle and syringe [not shown]. Other embodiments feature a chemotactic agent held in a sustained release vehicle, for example, a sustained release vehicle such as a biopolymer. Examples of biopolymers include gelatin, polyglyconic and polylactic acid derivatives. The biopolymers can be administered as microspheres or implants. The chemotactic agent is administered to a disease joint or an injured joint to direct mobilized and sensitized progenitor cells to the site where healing is desired.

The kit 11 includes instructions 21 and other materials and tools for making and using the elements contained therein. The instructions 21 will be described now in relationship to the method of using the kit 11.

The instructions 21 set forth a method of promoting joint complex healing. The method comprises the step of administering an effective amount of a bone marrow (BM)-derived vasculogenic progenitor cell mobilization factor to an animal or human exhibiting joint injury or joint disease. And, the method comprises the step of administering, concurrently to the mobilization factor, an effective amount of a progenitor cell sensitizing factor. The mobilization factor and sensitizing factor are reconstituted from the compounds in the first vial 15 and withdrawn from the first vial 15 with syringe 19. Syringe 19 is used to inject an effective amount of the mobilization factor and sensitizing factor subcutaneously, intraperitoneal, or intramuscularly into individual or animal to mobilize progenitor cells and sensitize the progenitor cells to one or more chemotactic agents present at the site of joint injury or joint disease.

The chemotactic agent is reconstituted from the powder held in second vial 17 and administered to the site of joint injury or joint disease. The site may have naturally occurring chemotactic agents and make the administration of the reconstituted chemotactic agent optional.

The use of a combination of mobilization factors and sensitizing factors improves healing of joint injuries and joint disease over healing exhibited by the use of either factor separate and apart from the other.

### EXAMPLES

Mice and Injury Model: All experiments are performed in accordance with the IACUC guidelines. C57BL/6J wild-type mice aged 8-12 weeks are purchased from Jackson Laboratories (Bar Harbor, Me.). Mice are randomized to receive one of #1) no injury; #2) full thickness partial transection of the medial collateral ligament of the knee; #3) full thickness partial excision of the medial meniscus of the knee; #4) articular medial intercondylar femoral osteotomy of the knee; and #5) full thickness partial transection in the patellar tendon. These injury models resemble the bone, cartilage, tendon, and ligament injury patterns common in humans.

Treatment Groups: Mice in each of the 4 experimental groups are randomly assigned to receive once daily one of: #1) saline i.p. injection; #2) AMD3100 (10 mg/kg, i.p.; PLERIXAFOR®; Genzyme Corp., Cambridge, Mass.) injection; #3) Teriparatide (0.285 mcg/kg, i.p.; FORTEO®; Eli Lilly and Company, Indianapolis, Ind.); or #4) AMD3100 (10mg/kg, i.p.; PLERIXAFOR®; Genzyme Corp., Cambridge, Mass.); and teriparatide (0.285 mcg/kg, i.p.; FORTEO®; Eli Lilly and Company, Indianapolis, Ind.).

Further experimental groups can be made with mice randomly assigned to receive one dose of SDF-1 is administered in an amount ranging from 1.00 ng to about 100 ng or one dose of saline by direct injection to soft tissue in and around the injured joint or disease joint.

Isolation of Mononuclear Cells (MNCs) from Peripheral Blood and Bone Marrow: Peripheral blood (PB) is harvested from mice at baseline, 7, 14, and 21 days post-wounding 1-hour following treatment with AMD3100, PTH, AMD3100+PTH, or sterile saline. BM is flushed from mouse long bones using PBS/10% FBS/5% EDTA, as previously described. Mononuclear cells (MNCs) from the peripheral blood and BM are isolated by density gradient centrifugation using Histopaque 1083 (Sigma-Aldrich; St. Louis, Mo.).

Flow Cytometry and Isolation of Progenitor Cells: For characterization by flow cytometry, PB MNCs are labeled with rat anti-mouse antibodies (fluorescein isothiocyanate conjugated Sca-1, allophycocyanin-conjugated c-kit, strepavidin-PE-conjugated-Cy7) (BD Bioscience; San Jose, Calif. and Miltenyi Biotech). All antibodies are titrated and optimized for appropriate detection. Samples are collected using a BD FACSCaliber flow cytometer (Becton-Dickinson; Franklin Lakes, N.J.) and analyses are performed with FlowJo 8.0 software (TreeStar Inc.; Ashland, Oreg.).

PCs are isolated from BM-MNCs by magnetic cell separation using a commercially available mouse lineage depletion kit (MACS®, Miltenyi Biotec, Inc.; Auburn, Calif.). Using this kit, lineage positive cells are removed, leaving an enriched lineage negative (lin−) cell population.

Isolated lin− cells are stained with FITC-Sca-1, APC-c-kit and sorted using a Dako MoFlo cell sorter (Dako Colorado Inc.; Fort Collins, Colo.). Enriched lin−/Sca−1+/c-kit+ cells (L−S+C+) are seeded onto 24-well plates (1,000 cells/well) (Corning Costar, Lowell, Mass.) and expanded in StemSpan Serum-Free media (Stem Cell Technologies; Vancouver, BC, Canada) supplemented with thrombopoietin [TPO: 20 ng/mL], stem cell factor [SCF: 100 ng/mL], interleukin-6 [IL-6:20 ng/mL], vascular endothelial growth factor [VEGF: 50 ng/mL], and Flt-3 [100 ng/mL] (Peprotech; Rocky Hill, N.J.). The L-S+C+ cell population is heterogenous, but enriched for vasculogenic PCs (Tepper O M, Carr J, Allen R J, Jr., Chang C C, Lin CD, Tanaka R, Gupta S M, Levine J P, Saadeh P B, Warren S M: Decreased circulating progenitor cell number and failed mechanisms of stromal cell-derived factor-1 alpha mediated bone marrow mobilization impair diabetic tissue repair. Diabetes 2010;59:1974-1983, the contents of which are hereby incorporated by reference in its entirety). Supplemented StemSpan is considered vasculogenic PC growth medium. All assays are performed on primary cultured PCs following 7 days of expansion.

Chemotaxis Assay: PC migration is measured using a modified Boyden chamber assay as previously described. Briefly, SDF-1α (100 ng/mL), PDGF-BB (100 ng/mL) or FBS (control) in vasculogenic PC growth medium or standard cell growth media is placed in the bottom of a 24-well plate. Cells (5×104)±AMD3100 (5-50 ng/mL)±rhPTH (5-50 ng/mL) is seeded onto fibronectin-coated (5 μg/cm2) transwell inserts. After 20 hours cells are harvested from the bottom chambers, washed, and centrifuged. Cell pellets are frozen at −80° C. Frozen cells are resuspended in CyQuant Green Fluorescent dye (Invitrogen) and the relative fluorescence is measured using a Synergy TM HT microplate reader (BioTek; Winooski, Vt.).

Adhesion Assay: Adhesion of PCs is measured in AMD3100 (5-50 ng/mL)±rhPTH (5-50 ng/mL). PCs (1×105 cells/chamber) are added to 4 well chamber slides (Fisher Scientific; Pittsburgh, Pa.) coated with fibronectin (5 μg/cm2) (Sigma) and incubated at 37° C. for 2 hours. Following incubation, non-adherent cells are removed before adherent cells are fixed with 1% paraformaldehyde. Adherent cells are stained with DAPI (4′,6 diamidino-2-phenylindole) (VectaShield; Vector Laboratories, Burlingame, Calif.) and viewed on an Olympus BX51 epifluorescent microscope. Adobe Photoshop CS3 (Adobe Systems; San Jose, Calif.) is used to quantify the number of cells/random high-powered field (hpf) under 100× magnification.

Proliferation Assay: Proliferation of PCs is measured using BrdU (5-Bromo-2′ deoxyuridine) labeling and fluorescent detection (Synergy TM HT microplate reader: BioTek; Winooski, Vt.). Proliferation is compared in media containing AMD3100 (5-50 ng/mL)±rhPTH (5-50 ng/mL).

Histology and Immunofluorescence: Bone, tendon, cartilage and ligaments are harvested on days 14, 21, and 28 for analysis. Frozen sections are stained with rat anti-mouse CD31 (PECAM; BD Biosciences) primary antibody and goat anti-rat IgG secondary (Alexafluor 594; Invitrogen). Control samples are prepared without primary antibody. Slides are mounted with DAPI (Sigma) and viewed on an Olympus BX51 epifluorescent microscope. DAPI is used to determine the sample outline; whereas, immunofluorescent CD31 staining is used to identify vascular structures (red staining) within the sample. Dual filter images are superimposed to illustrate wound architecture and vascular staining. Adobe Photoshop CS3 is used to segment and quantify positive CD31 staining The vascular density of mouse wounds is determined by quantifying the total area of CD31+ staining (red) per megapixel (1×106 pixels square area) of wound stained. Paraffin sections are stained with hematoxylin and eosin (H&E) to compare wound architecture between treatment groups as well as to confirm the full-thickness nature of the punch biopsies.

Statistical Analysis: Data is presented as mean standard error of the mean. A one way ANOVA with post-hoc Tukey Kramer is used for comparison of wound closure rates, cPC number, and vascular staining between all groups studied. A Student's t test is used for comparison between groups for the functional assays. Statistical significance is considered to be p<0.05. The number of mice per treatment group is determined using G*Power (G*Power®, Melbourne, Australia) to provide a power greater than 0.80.

### DISCUSSION

Our evidence suggests that vascularization plays a significant role in tissue healing. The present application, encompasses, in part, an endogenous strategy to improve bone, tendon, cartilage and ligament healing by promoting revascularization. In the present study, we show that endogenously mobilizing stem cells and concomitantly enhancing their trafficking yields a remarkable increase in bony healing. While systemic AMD3100 administration resulted in 59.7% bony ingrowth and PTH alone resulted in 56% bony ingrowth, together a synergistic effect of 90.6% bony regeneration was achieved; this was associated with significantly increased numbers of cPCs and CD31 staining in the trephine defect. Our results suggest that mobilized vasculogenic PCs increase new blood vessel formation at the site of injury and substantially increase bony regeneration. Since the effect of combining AMD3100 and rhPTH was synergistic, it was clear that rhPTH was not just acting through a local proliferative osteoprogenitor effect, but was effectively improving PC trafficking. Our in vitro adhesion assay results strongly support that the nature of systemic rhPTH synergistic effect was through improved cPCs trafficking and tubule formation.

These findings reinforce that tissue healing is multifactorial. The present inventors have found that the combination of teriparatide (1-34 portion of PTH) and AMD3100 (PLERIXAFOR®) will enhance joint complex healing. Without wishing to be bound by theory, targeting two completely different pathways, both equally essential to bone and soft tissue growth, we will provide a level of healing not demonstrated before. Further, a major advantage of using these two drugs, aside from their biological efficacy, is that their safety in humans has already been established.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1. A method of promoting joint complex healing, comprising the steps:
a. administering an effective amount of a bone marrow (BM)-derived vasculogenic progenitor cell mobilization factor to an animal or human exhibiting joint injury or joint disease;
b. administering, concurrently to said mobilization factor, an effective amount of a progenitor cell sensitizing factor to mobilize progenitor cells and sensitize said progenitor cells to one or more chemotactic agents present at the site of joint injury or joint disease.

2. The method of claim 1 wherein said mobilization factor is selected from the group consisting of CXCR4 agonist and partial agonists, granulocyte stimulating factor (G-CSF), granulocyte-macrophage stimulating factor (GM-CFS), Interleukin-1 (Il-1), Interleukin-3 (Il-3), interleukin-8 (Il-8), PIXY-321 (GM-CSF/Il-3 fusion protein), macrophage inflammatory protein, and growth related oncogene and agents and factors that modify the expression of the above factors.

3. The method of claim 2 wherein said CXCR4 agonists and partial agonists is AMD3100.

4. The method of claim 1 wherein said sensitizing factor is selected from the group consisting of parathyroid hormone and subunits of such hormone, NEL-like molecule-1, calreticulin, and closely related molecules, and agents and factors that modify the expression of the above factors.

5. The method of claim 4 wherein said parathyroid hormone and subunits thereof is recombinant human parathyroid hormone.

6. The method of claim 1 further comprising the step of administering at least one chemotactic factor to the area of the joint injury or joint disease.

7. The method of claim 6 wherein the chemotactic agent is selected from the group consisting of stromal cell derived factors, transforming growth factors, bone morphogenic proteins, fibroblast growth factors, vascular endothelial growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors.

8. The method of claim 1 wherein said mobilization factor and sensitization factor are co-administered in by subcutaneous, intraperitoneal or intravenous injection.

9. The method of claim 6 wherein said chemotactic agent is administered by injection.

10. As article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize said progenitor cells to one or more chemotactic agents present at the site of joint injury or joint disease.

11. The dosage form of claim 10 wherein said effective amount of said mobilization factor and said effective amount of said sensitizing factor are lyophilized and held in a vial for reconstitution.

12. The dosage form of claim 10 wherein said effective amount of said mobilization factor and said effective amount of said sensitizing factor are held in a package with an effective amount of a chemotactic agent which chemotactic agent is administered to a disease joint or an injured joint to direct mobilized and sensitized progenitor cells to the site where healing is desired.

13. The dosage form of claim 12 wherein the chemotactic agent is selected from the group consisting of transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors.

14. The dosage form of claim 12 wherein said chemotactic agent is lyophilized and held in a vial for reconstitution.

15. The dosage form of claim 12 wherein said chemotactic agent is administered by direct injection to soft tissue in and around the injured joint or disease joint.

16. The dosage form of claim 12 wherein said chemotactic agent is held in a sustained release vehicle.

17. The dosage form of claim 15 wherein said sustained release vehicle is a biopolymer.

18. The dosage form of claim 17 wherein said biopolymer is selected from the group comprising gelatin, polyglyconic and polylactic acid derivatives.

19. The dosage form of claim 17 wherein said biopolymer is formed as microspheres containing said chemotactic agent.
